(19) United States
(12) Patent Application Publication
(10) Pub. No.: US 2007/0212366 A1
(43) Pub. Date: Sep. 13, 2007

(54) Use Of Mrp4-Inhibitors For The Treatment And/Or Prophylaxis Of Cardiovascular Diseases

(75) Inventors: Andreas Greinacher, Suderholz (DE); Heyo K. Kroemer, Neuerkirchen (DE); Gabriele Jedlitschky, Greifswald (DE)

Correspondence Address: MARSHALL, GERSTEIN & BORUN LLP, 233 S. WACKER DRIVE, SUITE 6300, SEARS TOWER, CHICAGO, IL, 60606, US

(73) Assignee: ERNST-MORITZ-ARNDT-UNIVERSITAT GREIFSWALD, Greifswald (DE)

(21) Appl. No.: 10/578,452

(22) PCT Filed: Nov. 5, 2004
(86) PCT No.: PCT/EP04/12554

§ 371 Date: Mar. 15, 2007

(30) Foreign Application Priority Data
Nov. 7, 2003 (DE) 103 52 511.4

(51) Int. Cl. A61K 39/00; A61K 31/519; A61K 31/4965

(52) U.S. Cl. 424/185.1; 514/252.16; 514/47; 514/49; 514/252.1; 514/262.1

(57) ABSTRACT

The invention relates to the use of inhibitors of the multidrug resistance protein 4 (MRP4) in thrombocytes for the treatment and/or prophylaxis of cardiovascular diseases.

The present invention relates to the use of inhibitors of the multidrug resistance protein 4 (MRP4) in platelets for the treatment and/or prophylaxis of cardiovascular diseases.

The treatment of cardiovascular diseases, such as cardiac infarction, stroke and other arterial vascular occlusions, and the primary and secondary prophylaxis of arterial thromboembolic complications is carried out in practice according to different approaches. For example with GPIIb/IIIa blockers in the treatment of acute coronary syndroms. Long-term therapy with oral platelet aggregation inhibitors has an important significance in the treatment of patients with arteriosclerosis both in the context of secondary and primary prevention. In particular, therapy with the cyclooxygenase inhibitor acetylsalicylic acid (ASA) may be mentioned. Therapy with “ADP receptor antagonists” has won increasing acceptance in medical practice. Influencing of ADP-mediated platelet activation has attained so great a clinical significance because ADP is one of the most important activator substances of platelet activation. The platelet activator ADP is released, for example, from activated platelets and red blood corpuscles. It induces the further activation and aggregation of platelets. Nonsteroidal anti-inflammatory drugs (NSAID), such as diclofenac or ibuprofen, inhibit the ADP release which normally commences after activation of platelets. ADP receptor antagonists, such as, for example, clopidogrel, inhibit the binding of ADP to its receptor on platelets and thus ADP-induced platelet aggregation. Undesired effects, such as gastrointestinal side effects (e.g. diarrhea, nausea and vomiting), severe gastrointestinal hemorrhages, neutropenia or thrombocytopenia, which are observed with some ADP receptor antagonists, demand alternative treatment strategies. A further significant disadvantage of the substances employed hitherto is that for none of the substances is an antidote available. In the case of the necessity of an invasive intervention, such as an operation and in the case of hemorrhages, this leads to great problems.

It is therefore the object of the present invention to make available alternative medicaments for the primary and secondary prophylaxis of thromboembolic complications, in particular of cardiac infarction, stroke and other arterial vascular occlusions, or for the general treatment of cardiovascular diseases.

The object is achieved according to the invention by the use of inhibitors of the multidrug resistance protein 4 (MRP4) expressed in platelets.

In the context of the present invention, it was surprisingly found that MRP4 (also designated as the ABC pump or ABC transporter ABCC4) is expressed in human platelets and is located both on the extracellular membrane and intracellularly in the granules (transmitter storage granules). Transporter proteins which mediate the accumulation of ADP in the granules were unknown up to now. MRP4 has now been identified as a transporter for ADP, i.e. only by means of MRP 4 ADP is stored in the concentrations in the dense granules of the platelets which are necessary for the activation of further platelets in the case of release during platelet activation.

Since ADP stored in delta-granules plays a central role in the self-amplification of platelet activation, the inhibition of the MRP4 transporter protein is a novel approach for the treatment of the aforementioned diseases.

Owing to the inhibition of MRP4, the total amount of ADP in the platelets is reduced. By this means, one of the most important mechanisms of self-amplification of platelet activation is inhibited and thus also overshooting platelet activation. By means of this hitherto unused therapy principle, the formation of large clots in the arteries is inhibited and thus the formation of arterial vascular occlusions, which are causal for acute coronary syndrome, stroke and vascular occlusions in peripheral arterial occlusive disease.

From examinations of patients with genetic defects of the dense granules (synonym: delta-granules), it is known that their platelets cannot store ADP and therefore also cannot release it during platelet activation. These patients show only a low tendency to hemorrhage. This probably lies in the fact that ADP released from other cells can still bind to the platelets. By means of the therapy principle of inhibition of MRP4-mediated storage of ADP in the platelets described here for the first time, the phenotype of these genetically-related diseases is reached by use of medicaments. This therapeutic approach proposed in the context of the present invention is completely novel. None of the medicaments for the inhibition of platelet function previously available aims in its active principle at the reduction of the amount of ADP stored in platelets. This therapeutic approach is also safer than previously available therapeutic approaches of platelet function inhibition. From the therapy of patients having a congenital deficiency of ADP stored in platelets, it is known that the vasopressin analog DDAVP achieves a normalization of the tendency to hemorrhage within minutes. It likewise results from the principle of action that the ADP content of transfused platelets is not reduced by an MRP 4 inhibitor and thus, in contrast to GPIIb/IIIa inhibitors and in contrast to ADP receptor antagonists, transfused platelets can be employed effectively for the treatment of severe hemorrhages.

The present invention therefore relates to the use of one or more inhibitors of the multidrug resistance protein 4 (MRP4) in platelets for the production of a pharmaceutical preparation for the treatment of disorders of platelet function and for the treatment and/or prophylaxis of cardiovascular diseases. This includes the therapy, primary prophylaxis and/or secondary prophylaxis of acute coronary syndrome, angina pectoris, cardiac infarction, stroke or peripheral arterial occlusive disease, both before, during and after stent implantation in vessels.

MRP4 inhibitors proposed according to the invention are, for example, peptides, peptide analogs, peptidomimetics and cyclooxygenase inhibitors, especially nonsteroidal, antiinflammatory active compounds, which are either administered directly as an active compound, or as a “prodrug”, from which the active compound is formed by the endogenous metabolism. Another suitable active compound is, for example, dipyridamole.

According to one embodiment of the invention, amphiphilic organic, neutral or anionic compounds having a molecular weight of about 200 to 1000 daltons (Da) are used, which inhibit the MRP4-mediated transport of nucleotides (release of platelets). These include: dipyridamole, indomethacin, ibuprofen, inhibitors of organic anion transporters such as probenecid and sulfinpyrazone, phosphodiesterase inhibitors, in particular structural analogs of cyclic nucleotides such as sildenafil, trequensin, zaprinast, and the leukotriene receptor antagonist MK571 (3-(3-(2-(7-chloro-2-quinolinyl)ethenyl)phenyl)-((3-dimethylamino-3-oxopropyl)thio)methyl)thio)propanoic acid; cf. Jones et al. (1989) Can. J. Physiol. Pharmacol. 67, 17-28, Lalloo et al. BMC Med. 2 (2004) 16 and the publications cited in these articles).

The knowledge that MRP4 functions as an ADP transporter protein in the granules of human platelets can now be utilized in order to search for active compounds which inhibit this function of the protein MRP4.

The invention therefore also relates to a process for the identification of a substance which inhibits the ADP transporter protein MRP4 in platelets—i.e. of an active compound for the treatment of the aforementioned diseases—in which
a) the substance to be investigated is brought into contact with platelets in vivo or in vitro, a platelet activator is added and the change in the concentration of an activation marker in comparison to activated platelets which are not brought into contact with the substance to be investigated is measured (in vivo or in vitro), and
b) in membrane vesicles or granules comprising MRP4 which are likewise brought into contact with the substance to be investigated, the change in labeled (e.g. with radioactivity), absorbed cAMP or cGMP or labeled ADP is measured in comparison to membrane vesicles or granules (e.g. by measuring the radioactivity of absorbed [³H]cAMP or [³H]cGMP) which are not brought into contact with the substance to be investigated,
   the substance inhibiting the ADP transporter protein MRP4 in platelets if the substance in a) and/or b) in each case leads to a decrease in the particular measurement.

According to a particular embodiment, the afore-mentioned process additionally comprises a further step in which
c) the substance to be investigated is brought into contact with platelets in vivo or in vitro and the ADP concentration in the platelets is determined before and after,
   the substance inhibiting the ADP transporter protein MRP4 in platelets if the substance in a) and/or b) and/or c) in each case leads to a decrease in the particular measurement.

The aforementioned determinations under a) and b) or a) and b) and c) can of course be carried out in any desired sequence. For the initial screening, the determination mentioned under b) can also initially be carried out alone.

Alternatively, the process can also be carried out by carrying out step a) on platelet granule membranes (cf. Example 5).

Furthermore, for the further investigation of its activity the substance can be administered in one step
d) to an experimental animal or a subject, the ADP content of the platelets of the experimental animal or of the subject being determined before and after administration of the substance.

An ADP content decreasing after the or during the treatment points to an MRP4-inhibiting action of the investigated substance.

A substance which inhibits the ADP-transporter protein in platelets is understood within the context of the present invention as meaning a substance which is suitable for decreasing, i.e. completely or partly blocking, the ADP enrichment in granules. Here, these substances are not restricted to substances having a specific mechanism of action. The substances can be organic chemical compounds as well as oligopeptides or antibodies.

The term “multidrug resistance protein 4 (MRP4)” is understood in the prior art as meaning the human MRP4 gene product and its various isoforms, as well as analogs, homologs and orthologs in other species. These shall also be included according to the invention. The human gene which codes for MRP4 was initially partly cloned and is to be found on chromosome 13 (GenBank accession number U83660, Kool et al., Cancer Res. 57, (1997) 3537-3547).

For carrying out the process for the identification of MRP4 inhibitors, platelets from non-human sources, such as, for example, mice, rats, rabbits, primates, transgenic animals, knock-out animals can therefore also be used. The active compounds thus identified should preferably be tested subsequently with respect to their action on human platelets.

Using the present invention, furthermore a process for the production of a pharmaceutical composition for the treatment and/or prophylaxis of cardiovascular diseases, including the therapy, primary prophylaxis and/or secondary prophylaxis of acute coronary syndrome, angina pectoris, cardiac infarction, stroke or peripheral arterial occlusive disease, both before, during and after stent implantation in vessels, is made available, in which an aforementioned process for the identification of substances which inhibit the ADP transporter protein MRP4 in platelets is carried out and the substances identified in this way are formulated using pharmaceutically acceptable excipients and/or carriers.

The invention thus relates to the use of a substance obtained according to an aforementioned process for the identification of a substance inhibiting the ADP transporter protein MRP4 in platelets for the production of a pharmaceutical preparation for the treatment and/or prophylaxis of the aforementioned diseases.

Using the knowledge obtained according to the invention that MRP4 functions as an ADP transporter in granules of human platelets, alternative protocols for the therapy, primary and/or secondary prophylaxis of the aforementioned diseases are made available. By titration of the inhibition of the ADP uptake, individual therapy control is possible. By determination of the ADP content of the platelets, an individual measurement of the action of the medicament is possible. This has not been possible until now with the previous substances, e.g. ADP receptor antagonists. If the necessity of invasive intervention exists, the medicament effect can be antagonized by the vasopressin analog DDAVP and by the transfusion of platelets. This is not adequately possible in the acute phase with GPIIb/IIIa antagonists, as well as not in the present ADP receptor antagonists, as the transfused platelets are immediately inhibited by the medicament.

The present invention is subsequently explained with the aid of examples, but without being restricted thereto.

### EXAMPLES

### Example 1

The expression and intracellular location of MRP4 in human platelets was detected by immunoblotting and immunofluorescence microscopy and a functional assay. In immunoblots, the MRP4-specific antibody (rabbit) showed a strong signal at the expected molecular mass of approximately 170 kDa in homogenates of isolated platelets and indicated an MRP4 enrichment in membrane fractions, which were separated by means of a sucrose density gradient.

The MRP4 function, which was demonstrated using ³H-labeled cyclic GMP (cGMP), a known MRP4 substrate, could likewise be demonstrated in fractions of low-density which contained the most plasma membrane proteins, as well as in fractions of higher density, in which intracellular granules were present.

Immunofluorescence microscopy of platelets with the same antibodies showed an enrichment of MRP4 in intracellular granules and a weak signal on the plasma membrane.

In duplicate staining experiments, MRP4-positive granules differ from granules which were positively stained compared to P-selectin, which is mainly expressed on alpha-granules. The MRP4-positive granules, however, showed an accumulation of the fluorescence dye mepacrine, of which it is known that it is transported in delta-granules.

For the detection of expression in platelets, washed platelets are incubated on glass plates coated with collagen and adhere to these. Alternatively to collagen, any desired other substances can be taken which bind to glass or plastic surfaces and bind to the platelets, or the platelets are directly bound to a carrier surface. The platelets are fixed, e.g. with 1% paraformaldehyde solution, and the cell membrane is opened by means of chemicals, e.g. saponin, such that non-membrane-permeable substances (antibodies, receptor binding partners) can also bind to intracellular structures. Then, the platelets are either incubated with a direct fluorescence-labeled antibody to MRP4, or with a nonlabeled antibody, and in a second step with a labeled secondary antibody which recognizes the primary antibody, e.g. goat anti-rabbit FITC. The location of the antibody binding is made possible by coincubation with a monoclonal or polyclonal antibody having known specificity for marker proteins of the platelet granules. The evaluation of the staining is carried out using fluorescence microscopy.

Alternatively, the platelets are fixed and incubated with directly or indirectly gold-labeled antibodies to MRP4 and the binding of the antibodies to structures of the dense granules is detected.

In order to confirm the location further, we have investigated the binding of anti-MRP4 antibodies to platelets which were obtained from a patient having Hermansky Pudlak syndrome. In this patient, the dense granules of the platelets are not present on account of a genetic disease, but the other platelet organelles (alpha granules, lysosomes) are preserved. Using the methods described above, it was seen that the platelets which specifically lack the dense granules express no MRP4 intracellularly, but express MRP4 on the platelet surface. The location is thus unequivocally confirmed.

For the immunoblot, platelets are washed in physiological saline solution, solubilized using standard methods and the lysate is separated according to molecular weight on a gel according to standard methods. The proteins in the gel are transferred to a nitrocellulose membrane according to standard methods by applying a voltage gradient. The membrane is then incubated with the primary or secondary labeled antibodies to MRP4 and the binding of the antibodies is visualized by: fluorescence, chemical reaction (horseradish peroxidase reaction) or by radiolabeling.

### Example 2

Measurement of the MRP4 Function in Platelet Membranes

The preparation of the membrane vesicles and the transport measurements are carried out following the methods described in detail for MRP1 and culture cells by Keppler et al. (Methods Enzymol. 292 (1998) 607-616).

Washing of the Platelets

About 30 ml of liquid from the platelet concentrates are transferred to a Falcon tube and centrifuged for 10 min at 4° C. at a centrifugation speed of 1300 g. After pouring off the supernatant, the pellet is resuspended in 1 ml of solution B (9.3 ml of NaCl solution (0.9%), 0.5 ml of solution A (5 g of EDTA, NaCl solution (0.9%) to 100 ml), 0.2 ml of bovine serum albumin solution (22%)) and made up to a volume of 30 ml with solution B. A further centrifugation at 1300 g for 10 min at 4° C. is carried out. The pellet obtained is resuspended in 1 ml of solution C (NaCl solution (0.9%) adjusted to pH 6.5 with 1 PBS pH 7.2) and made up to a volume of 30 ml with solution C. This step is repeated three times, the platelets only being resuspended in the last washing. Storage then takes place in aliquots of 100 μl at −20° C. In order to minimize the erythrocyte concentration of the platelet concentrate, the loose, white pellet of platelets is removed after the first centrifugation using a Pasteur pipette and thus separated from the more strongly depositing erythrocytes.

Cell Disruption

Ultrasound

This disruption method is used on the washed platelets. The stock solutions of the protease inhibitors leupeptin, aprotinin and phenylmethylsulfonyl fluoride are in each case added in the ratio 1:1000 to the cell suspension of a volume of around 500 μl. The disruption is carried out using a Sonopuls apparatus in a cycle of 5 10 s at a force of 50% with cooling breaks of 2 min between the individual cycles. Subsequently, centrifugation is carried out at 100,000 g for 30 min at 4° C. The pellet obtained is resuspended in 50 μl of 5 mM Tris solution, pH 7.4 and the protein content is determined.

Freezing/Thawing

The washed platelets are present in a cell suspension having a total volume of around 500 μl. After addition of the stock solutions of the protease inhibitors aprotinin, leupeptin and PMSF in the ratio 1:1000, disruption is carried out by freezing in liquid nitrogen and rethawing in a water bath at 37° C. This cycle is carried out a total of five times. Subsequently, centrifugation at 100,000 g is carried out for 30 min at 4° C. The pellet obtained is resuspended in 50 μl of 5 mM Tris solution, pH 7.4, and the protein content is determined.

Membrane Preparation

Platelets

The suspension obtained by cell disruption with the aid of freezing and thawing is free of cytosolic proteins and thus contains the plasma membranes and further cell organelles, such as mitochondria and granules. It is worked up as follows to give membrane vesicles.

The membrane suspension is transferred to a Dounce-Potter (tight) and diluted to approximately twice the volume with Tris-sucrose buffer. Subsequently, the addition of the protease inhibitors leupeptin and aprotinin is carried out in the volume ratio of the stock solution to the suspension of 1:1000. These protease inhibitors are competitive inhibitors whereas PMSF, which was already added during the disruption, inhibits irreversibly.

Afterward, 30 Potter strokes are carried out such that approximately 2 strokes per minute take place.

Membrane Preparation without Sucrose Gradient

After the homogenization process, two thirds of the suspension are centrifuged at 100,000 g at 4° C. for 30 min. The pellet is resuspended in a volume of Tris-sucrose buffer corresponding to 4 times the pellet volume, again transferred to the Dounce-Potter (tight) and homogenized using 20 Potter strokes. Finally, the suspension is sucked up 20 times by means of a 27G needle in order in this way to produce membrane vesicles which, in particular, are aligned inside out. Storage takes place in liquid nitrogen as 100 μl aliquots.

Membrane Preparation with Sucrose Gradient

After the homogenization process, a third of the suspension is added to a sucrose gradient. The 4 ml-capacity tubes which belong to the swing-out rotor SW55 of the Beckman centrifuge are loaded as follows. First, 1.5 ml of a 60% sucrose solution in 5 mM HEPES, pH 7.4 (m/m) are added. Then 1.5 ml of a 30% sucrose solution in 5 mM HEPES, pH 7.4 (m/m) are added. 0.5 ml of the membrane suspension is layered over these two sucrose solutions. The subsequent centrifugation with the swing-out rotor is carried out at 200,000 g for 60 min at 4° C.

Transport Measurement

The basis of the method used is membrane vesicles which are formed by means of shear forces during sucking up with a needle which is as thin as possible. The manner of preparation of the membrane vesicles always also produces a certain proportion (around 30%) of inside-out membrane vesicles. The particular feature of the inside-out membrane vesicles is that the membrane is aligned the other way round to the plasma membrane. An outward transporter of the plasma membrane would transport inward in an inside-out vesicle. Since the MRP4 is furthermore present in the plasma membrane, inside-out vesicles offer the possibility of a function test. Moreover, in platelets and other cells vesicles exist in the cytoplasm, which are formed by constrictions of the plasma membrane and are likewise aligned inside-out and consequently could be absorbed by transport processes.

A tritium-labeled substrate which can be transported in an ATP-dependent manner is added to the inside-out vesicles. By measurement in the presence of AMP and ATP (ATP-maintaining creatine kinase), the ATP-dependent inward transport is measurable at the same time, from which the AMP value, which reflects diffusion processes, is subtracted.

The substances found in the inside-out vesicles are added to a filter having a diameter of 0.22 μm such that excess radioactivity is removed by washing. The result is detected by means of counting of the radioactive disintegrations on the filters (see FIG. 1).

The transport experiments were carried out using [3H]-cGMP (tritium-labeled cGMP).

Per batch, 100 μg of protein of the membrane vesicle suspension are diluted to 55 μl with Tris-sucrose buffer. In a second batch, 7.5 μl of creatine kinase, 6.8 μl of ATP or AMP and 7.5 μl of a [³H]-cGMP stock dilution, are diluted with Tris-sucrose buffer in the ratio 2:5 for the [³H]-cGMP (1 μCi/μl). The final concentration of the [³H]-cGMP corresponds to 4 μM with a radioactivity of 2.7 μCi/75 μl.

The 55 μl of the membrane suspension and the radioactive batch are simultaneously warmed at 37° C. for one minute in a thermoshaker, before 20 μl of the radioactive batch are pipetted into the membrane suspension and the transport experiment is thus started. During the incubation in the thermoshaker at 37° C., 20 μl samples are in each case taken after 1, 10 and 20 min. The samples removed are pipetted into 1 ml of ice-cooled tris-sucrose solution, the total volume immediately being added to a nitrocellulose filter. The free radioactivity is removed by rinsing with 5 ml of cold tris-sucrose solution under applied vacuum. The individual filters are transferred to vials and 10 ml of scintillation fluid (e.g. Roth, Zinsser) are added. This fluid converts the radioactivity present into an analyzable light signal, which can be measured as DPM (decays per minute=disintegrations per minute) using the Wallac 1409 Liquid Scintillation counter and a special tritium counting program.

Calculation

| [³H]-cGMP: | 8.8 Ci/mmol = 8.8 μCi/nmol | | |
|---|---|---|---|
| | 1 μCi ≡ 2.2 | 10⁶ DPM | |
| | 1 nmol ≡ 8.8 | 2.2 | 10⁶ DPM |

| | |
|---|---|
| | 0.05 pmol ≡ 1000 DPM |
| Protein: | 100 μg/75 μl |
| | 26.7 μg/20 μl (sample volume) |
| based on 1 mg of protein: | 1000 DPM ≡ 1.87 pmol/mg |

### Example 3

Inhibition of Transport:

In the test of potential inhibitors, these are added to the incubation in various concentrations together with the labeled substrate. Identification as an inhibitor is provided if the transport rate of the labeled substrate in multiple determinations is statistically significantly lowered (e.g. Students T test) compared to the control incubation without inhibitor.

### Example 4

Identification of MRP4-Inhibiting Substances by Measurement of the Platelet Function

A. Platelet Aggregation According to Born

The platelet function after inhibition of the MRP4 transporter can be measured by platelet aggregation according to Born (Platelets. In: Human Blood Coagulation, Haemostasis and Thrombosis, 2nd Ed. Biggs, Editor Blackwell Scientific Publications, London 1976, pp. 168-201). Here, platelets are added to a platelet activator and the change in the concentration of an activation marker is measured. The experiment is repeated under identical conditions, the platelets, however, being brought into contact beforehand with a substance to be investigated before the platelet activator is added. Alternatively, an experimental animal or a human subject is treated with the MRP4-inhibiting substance and the platelets obtained from this experimental animal or human subject are investigated using the same method and either compared with platelets of nontreated experimental animals or nontreated subjects or with the platelets of the experimental animal or of the human subject from before the treatment. The measurements obtained in both experiments are compared, the substance indicating MRP4 inhibition if the addition of the test substance compared to the platelet aggregation test without test substance leads to a decrease in the particular measurement.

Below, the principle of platelet function investigation by means of aggregometry according to Born is described.

| | |
|---|---|
| Measurement | turbidimetry |
| method: | |
| Apparatus: | 4-channel aggregometer “APACT 4” |
| | (Laborgeräte + Analysensysteme |
| | Vertriebsgesellschaft mbH, Ahrensburg, Germany) |
| Inducers: | ADP, collagen, ristocetin, adrenalin |
| Procedure: | |

1. Inducers:

The inducers are present as lyophilisates and are prepared according to the instructions of the manufacturers and can be stored at below −20° C. in aliquots until use.

The following inducer concentrations (final concentrations) are used:
ADP (2.5 μmol/l, 5 mmol/l,
10 mmol/l, 20 mmol/l)
collagen (1 μg/ml, 4 μg/ml)
ristocetin (0.5 mg/ml; 1.5 mg/ml)
adrenalin (5 μmol/l, 10 μmol/l)

The inducers are thawed shortly before use, vigorously mixed (vortexer) and stored on ice at 4° C. during use (adrenalin stores at room temperature during the procedure).

2. Obtainment of Platelet-Rich Plasma (PRP) and Platelet-Poor Plasma (PPP):

PRP is first obtained from a healthy blood donor by means of differential centrifugation of a citrate-anticoagulated, freshly taken whole blood sample (20 min, 120 g). The donor should have taken no medicaments adversely affecting platelet function for 10 days before taking blood.

The PRP is transferred to a clean poly tube and is adjusted to a platelet count of 300,000/μl with PPP from the same donor, which is obtained from PRP by high-speed centrifugation (5 min, 860 g). The adjusted PRP is stored at 37° C. until use and the tube is sealed with Parafilm.

3. Apparatus Settings and Measurement:

a) Settings on the APACT

600 sec. measuring time, stirrer speed 1000 rpm, temperature of the heating block 37° C. Heating time of the apparatus about 10 to 15 min.
   b) Calibration of the Apparatus Using PPP
180 μl of PPP are mixed with 20 μl of 0.9% NaCl solution pH 7.2 in a cuvette provided with a stirrer magnet, placed in the measuring channel and measured (corresponds to 100% light transmission).
   c) Calibration of the Apparatus Using PRP
180 μl of PRP are mixed with 20 μl of 0.9% NaCl solution pH 7.2 in a cuvette provided with a stirrer magnet, placed in the measuring channel and measured (corresponds to 0% light transmission).
   d) Measurement
A clean cuvette provided with a stirrer magnet is again filled bubble-free with 180 μl of PRP. The measuring channel is started; the recording of the measurement of the light transmission can be monitored on the PC. After about 1 min, 20 μl of one of the abovementioned inducers are pipetted into the PRP-filled cuvette (bubble-free, do not remove the cuvette from the measuring channel; take care that the inducer does not remain suspended at the edge of the cuvette!). The course of the curve is recorded continuously. The light transmission (%) of the PRP increases over time with the increase in the aggregated platelets.
The cuvette is removed from the measuring channel after the end of the measuring time. The measurements are then repeated, as described under b) to d), for each further inducer. For the measurement of the spontaneous aggregation of the platelets, instead of the inducer, 20 μl of physiological NaCl solution are pipetted into the PRP and the measurement is carried out.
   4. Quality Control:

The measurement of the function of the platelets of a healthy normal subject serves as a control of the condition of the inducers and the functionability of the aggregometer. The measurement of the platelet function of, for example, subjects having V.a. thrombocytopathy is carried out subsequently.

The potential MRP4 inhibitors to be investigated are added—as mentioned above—to the cuvettes before addition of the respective inducers.

For the investigation of whether the substance to be investigated specifically acts on the MRP4 transporter protein, the vesicle transport studies described in Example 3 are carried out.

B. Expression of Activation Markers in Flow Cytometry

The platelet function after inhibition of the MRP4 transporter can furthermore be determined by expression of activation markers (e.g. PAC-1, CD 63, P-selectin) in flow cytometry.

Flow cytometry is a process for the characterization of cells by means of the size and granularity. It moreover serves for the quantitative determination of surface molecules and intracellular proteins with the aid of fluorescence-labeled antibodies.

The labeled cells present in suspension are individually led past an argon laser (cf. FIG. 2). The fluorescence and scattered light resulting therefrom is detected by various photodetectors. The light scattered by the cell in the direction of the laser beam is absorbed by the FSC detector (forward scatter, FSC) and gives information about the size of the measured cell. The sideward scattered light (sideward scatter, SSC) correlates with the granularity of the cell. Additional characteristics of the cells, such as, for example, the expression of one or more surface molecules, are recorded by means of various further detectors. For each individual cell, the intensity and color of the fluorescence is detected by a computer. The signals of the photodetectors are amplified by means of a photomultiplier. The analysis of the corresponding data is carried out with the aid of the computer program WinMDI 2.8.

Activated and resting platelets differ morphologically from one another. Activated platelets are distinguished by the formation of microparticles and large platelet aggregates, which leads to a shift in the platelets in the FSC/SSC (Matzdorff et al. 1998, J. Lab. Clin. Med. 131(6): 507-17).

Resting platelets, on the other hand, exhibit a uniform size and therefore form a defined scatter diagram on consideration of size and granularity in the flow cytometer. In addition to the change in the shape of the platelets, surface exposure of P-selectin occurs.

For fixing, 100 μl of platelet suspension are pipetted into 1 ml of formaldehyde solution. After two hours to at most four days, the platelets are washed twice with FACS flow for staining (1700 g, 5 min). The pellet is resuspended in 500 up of FACS BSA mixture and 25 up of the sample are incubated in the dark for 30 min at room temperature with 10 μl of antibody (P-selectin, mouse, unlabeled). Subsequently, the batch is washed once with FACS flow. After aspirating the supernatant, 10 μl of secondary antibody (anti-mouse, FITC-labeled, diluted 1:30) are added to the platelets. The samples are incubated in the dark at room temperature for 30 min. For the measurement, the platelets are washed once and then taken up in 1 ml of FACS flow.

The measurements are carried out analogously to the investigation of platelet aggregation according to Born (see under A) by platelet activation in each case in comparison with and without substance to be investigated, the substance indicating an MRP4 inhibitor if the addition of the test substance compared to the measurement without test substance leads to a lower platelet activation and thus to a shift in the FSC/SSC ratio in the direction FSC.

For the investigation of whether the substance to be investigated specifically acts on the MRP4 transporter protein, the vesicle transport studies described in Example 3 are carried out.

C. Lumiaggregometry

The platelet function after inhibition of the MRP4 transporter can furthermore be determined by lumiaggregometry. Here, the release of ADP/ATP is measured in lumiaggregometry by means of luciferin-luciferase test (cf. Mondoro et al., Blood 96 (2000) 2487-2495 and White et al., Thromb. Haemost. 67 (1992) 572).

The measurements are carried out analogously to the investigation of platelet aggregation according to Born (see under A) or flow cytometry (cf. under B) by platelet activation in each case in comparison with and without substance to be investigated, or of experimental animals or human subjects treated with the substance in comparison to experimental animals or human subjects not treated with the substance, the substance indicating an MRP4 inhibition if the addition of the test substance compared to the measurement without test substance leads to a lower platelet activation and thus to a lower luminescence value.

The experiment is repeated under identical conditions, the platelets, however, being brought into contact beforehand with a substance to be investigated.

The measurements obtained in both experiments are compared, the substance indicating an MRP4 inhibition if the addition of the test substance compared to the lumiaggregometry without test substance leads to a decrease in the release of ADP/ATP. In addition to lumiaggregometry, other processes for quantification of ADP in platelets can also be used, such as, for example, the extraction of ADP and detection using the luciferin-luciferase method, or quantification using chromatographic processes, such as HPLC.

For investigation of whether the substance to be investigated acts specifically on the MRP4 transporter protein, the vesicle transport studies described in Example 3 are carried out.

### Example 5

Involvement of MRP4 in ADP Transport in Delta-Granules of Platelets

[³H]ADP Transport in Vesicular Fractions of Platelets.

In order to determine whether ADP can be a substrate for MRP4-mediated, ATP-dependent transport, the uptake of [3H]ADP (1 μM) in platelet membrane vesicles (crude membranes) in the presence of 0.4 mmol/l of ATP was measured over a period of 2 minutes. As shown in FIG. 3, a time-dependent increase in the vesicle-associated [³H]ADP at a rate of 6.74±1.9 μmol mg protein⁻¹ min⁻¹ (mean value±standard deviation of three different experiments with determination in triplicate) was observed. If ATP was replaced by 5′-AMP or the non-hydrolyzable ATP analog AMPPNP, which was customarily used in control incubations in order to calculate the ATP-dependent component of the transport, approximately a three to four-fold higher background binding of the [³H]ADP to the membranes was observed, which increased only slightly with time (not shown). This lower, nonspecific binding of [³H]ADP in the presence of ATP in comparison to 5′-AMP is probably to be attributed to the presence of unlabeled ADP, which competes with ³H-labeled ADP and dilutes the same. The source of this unlabeled ADP could be ADP contamination in the commercially obtainable ATP and the formation of ADP by ATP hydrolysis. In order to show the ATP dependence and simultaneously to ensure identical initial ADP concentrations, the transport was measured in the presence of ATP with or without 1 mM orthovanadate, an inhibitor of ATP hydrolysis. A time-dependent increase in the vesicle-associated radioactivity in the presence of ATP was observed, in spite of the fact that [³H]ADP was diluted by simultaneous formation of unlabeled ADP. This shows that there is an active intercalation which counteracts the dilution effect. The vesicle-associated radioactivity was significantly reduced in the presence of ATP plus orthovanadate in spite of the fact that the inhibition of ADP formation should increase the binding of the labeled compound, which thus shows that the observed decrease is the inhibition of the active transport process. The dilution effect on [³H]ADP by formation of unlabeled ADP was also the reason for reducing the ATP concentration from the standard concentration (4 mM) to 0.4 mM.

In order to determine whether the observed difference in the [³H]ADP uptake by the vesicles in the presence and absence of orthovanadate reflects a transmembrane movement rather than binding to the membrane surface, the influence of high osmolarity was investigated. At a concentration of 1 mol/l of sucrose outside the vesicles, the rate of [³H]ADP transport in the absence of orthovanadate was considerably reduced, which shows active transport (FIG. 3B). In the presence of orthovanadate, 1 mol/l of sucrose only slightly influenced the [³H]ADP association to the vesicles. This shows that the amount of measured radioactivity represents the ratio of the [³H]ADP binding to the vesicle membrane, independently of the transmembrane transport. Furthermore, the vanadate-sensitive ADP accumulation in the dense granular fraction of the sucrose gradient could be detected and was inhibited by dipyridamole, MK571 and cGMP (Table 1).

Methods:

Transport of [³H]ADP. An ATP-dependent transport of [3H]ADP (1 μmol/l) in the membrane vesicles was measured by rapid filtration, as is described for [³H]cGMP, with the exception that the vesicles in incubation buffer supplemented with 0.4 mmol/l ATP, 10 mmol/l MgCl₂ are incubated in the presence or absence of sodium orthovanadate (1 mmol/l). In order to investigate the effect of the osmolarity of the extra-vesicular medium, the vesicles were preincubated for 45 minutes at 4° C. in a 1 mol/l sucrose-containing buffer or in standard incubation buffer which contained 250 mmol/l of sucrose.

TABLE 1
Inhibition of the [³H]ADP transport in dense platelet granules

| | Concentration | [³H]ADP transport |
|---|---|---|
| Substance/component | (μM) | (% of control) |
| None (control) | | 100 |
| Dipyridamole | 10 | 52.7 ± 5.2 |
| | 100 | 8.8 ± 0.4 |
| MK571 | 100 | 40.7 ± 3.9 |
| cGMP | 100 | 28.7 ± 2.3 |

Dense platelet vesicles (100 μg of protein) were incubated with [3H]ADP (1 μmol/l) in the presence of 0.4 mmol/l of ATP or 0.4 mmol/l of ATP+1 mmol/l of orthovanadate for two minutes, and the difference in the vesicle-associated radioactivity was calculated. The compounds listed were added to the incubations with and without orthovanadate in the concentrations indicated. The difference is indicated as the percentage of the control (mean value+standard deviation of 3 determinations). The control value of the vanadate-sensitive [³H]ADP transport in this experiment was 3.0±0.4 μmol/mg of protein⁻¹ after 2 minutes.

### DESCRIPTION OF THE FIGURES

FIG. 1:

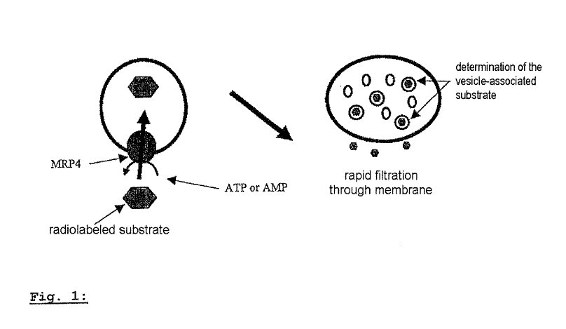

Principle of the transport measurement with inside-out membrane vesicles.

FIG. 2:

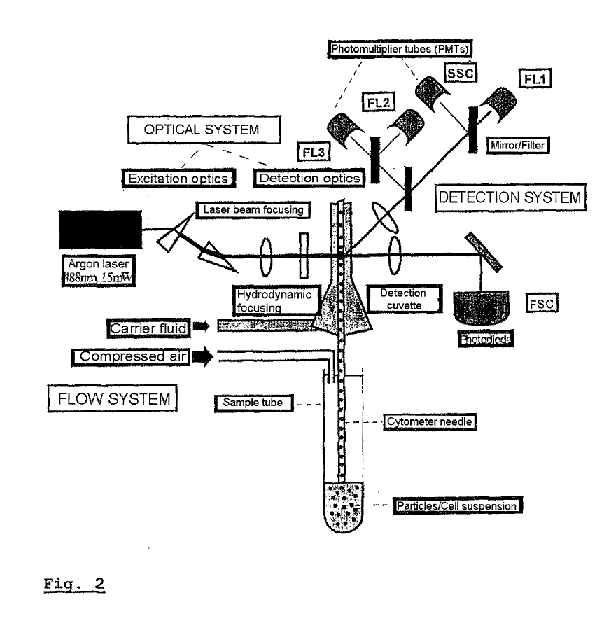

Construction of a flow cytometer (simplified schematic representation).

FIG. 3:

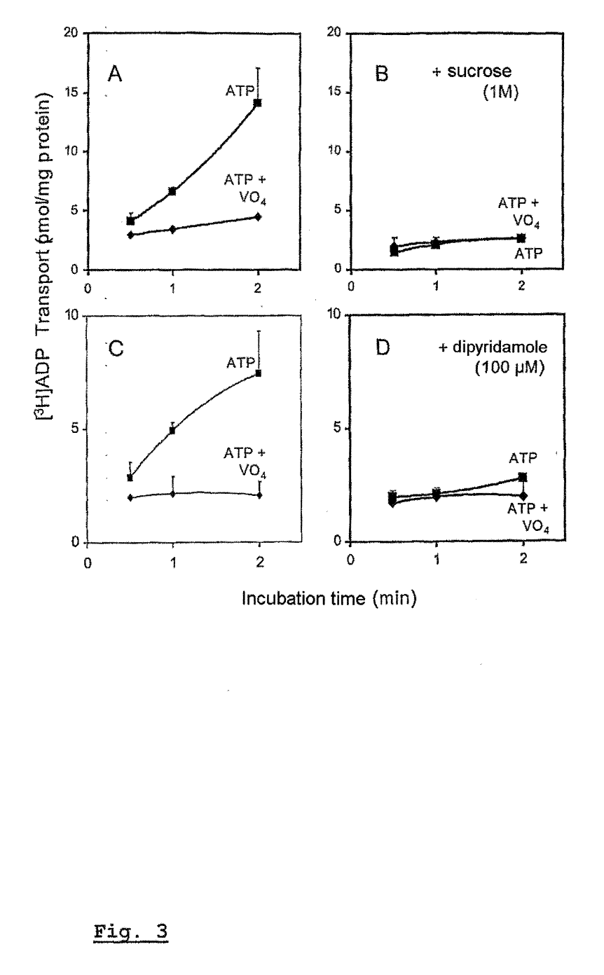

Transport of ADP in platelet membranes.

A-D: Crude membrane vesicles from platelets (100 μg of protein) were incubated with [³H]ADP (1 μmol/l) in the presence of 0.4 mmol/l of ATP (squares) or 0.4 mmol/l of ATP plus 1 mmol/l of orthovanadate (diamonds), and the vesicle-associated radioactivity was determined as in the section “Methods” (mean value±standard deviation, n≦3; 1 μmol mg of protein⁻¹=1724 DPM). A, B: Vesicles were preincubated for 45 minutes at 4° C. in standard incubation buffer which contained 250 mmol/l of sucrose (A), or in 1 mol/l of sucrose-containing buffer (B). C, D: ADP transport in the absence (C) or presence (D) of 100 μmol/l of dipyridamole.

1. Use of an inhibitor of the multidrug resistance protein 4 (MRP4) in platelets for the treatment and/or prophylaxis of cardiovascular diseases.

2. The use according to claim 1, characterized in that the treatment and/or prophylaxis of cardiovascular diseases is the therapy, primary prophylaxis and/or secondary prophylaxis of acute coronary syndrome, angina pectoris, cardiac infarction, stroke or peripheral arterial occlusive disease before, during and after stent implantation in vessels.

3. The use according to claim 1 and 2, characterized in that the active compound is an amphiphilic organic, neutral or anionic compound having a molecular weight of about 200 to 1000 daltons (Da), which inhibits the MRP4-mediated transport of nucleotides.

4. The use according to claim 3, characterized in that the active compound is dipyridamole, indomethacin, ibuprofen, inhibitors of organic anionic transporters such as probenecid and sulfinpyrazone, structural analogs of cyclic nucleotides such as sildenafil, trequensin, zaprinast (phosphodiesterase inhibitors) and of the leukotriene receptor antagonist MK571.

5. Process for the identification of a substance which inhibits the ADP transporter protein MRP4 in platelets, characterized in that
a) the substance to be investigated is brought into contact with platelets in vivo or in vitro, a platelet activator is added and the change in the concentration of an activation marker in comparison to activated platelets which are not brought into contact with the substance to be investigated is measured (in vivo or in vitro), and
b) in membrane vesicles comprising MRP4 or granules which are likewise brought into contact with the substance to be investigated, the change in labeled, absorbed cAMP or cGMP is measured in comparison to membrane vesicles or granules which are not brought into contact with the substance to be investigated,
the substance inhibiting the ADP transporter protein MRP4 in platelets if the substance in a) and/or b) in each case leads to a decrease in the particular measurement.

6. The process according to claim 5, characterized in that furthermore
c) the substance to be investigated is brought into contact with platelets in vivo or in vitro and the ADP concentration in the platelets is determined before and after,
the substance inhibiting the ADP transporter protein MRP4 in platelets if the substance in a) and/or b) and/or c) in each case leads to a decrease in the particular measurement.

7. The process according to claim 5 or 6, characterized in that a) and b) or a) and b) and c) are carried out in any desired sequence.

8. Process for the preparation of a pharmaceutical composition for the treatment and/or prophylaxis of cardiovascular diseases, characterized in that a process according to claims 4 to 7 is carried out and the substances identified are formulated using pharmaceutically acceptable excipients and/or carriers.

9. The process according to claim 8, characterized in that the treatment and/or prophylaxis of cardiovascular diseases is the therapy, primary prophylaxis and/or secondary prophylaxis of acute coronary syndrome, angina pectoris, cardiac infarction, stroke or peripheral arterial occlusive disease before, during and after stent implantation in vessels.

10. Use of a substance identified by the process of claims 4 to 7 for the treatment and/or prophylaxis of cardiovascular diseases.

11. The use according to claim 10, characterized in that the treatment and/or prophylaxis of cardiovascular diseases is the therapy, primary prophylaxis and/or secondary prophylaxis of acute coronary syndrome, angina pectoris, cardiac infarction, stroke or peripheral arterial occlusive disease before, during and after stent implantation in vessels.
